(19) United States
(12) Patent Application Publication
(10) Pub. No.: US 2007/0244335 A1
(43) Pub. Date: Oct. 18, 2007

(54) Isolation of tetracycline derivatives

(75) Inventors: Evgeny Tsiperman, Beer-Sheba (IL); Sergei Fine, Qiriat-Arbaa (IL); Sofia Gorohovsky, Beer Sheva (IL); Slavik Yurkovsky, Kiryat Gat-Malkei (IL)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Teva Pharmaceutical Industries Ltd., Petach Tikva (IL)

(21) Appl. No.: 11/787,659

(22) Filed: Apr. 17, 2007

Related U.S. Application Data
(60) Provisional application No. 60/792,814, filed on Apr. 17, 2006.

(51) Int. Cl. C07C 237/26

(52) U.S. Cl. 552/205

(57) ABSTRACT

Provided is a process for the isolation of tetracycline derivatives.

### CROSS-REFERENCE TO RELATED APPLICATIONS

This application claims the benefit of the filing date of U.S. Provisional Patent Application No. 60/792,814 filed Apr. 17, 2006, the disclosure of which is hereby incorporated herein by reference.

### BACKGROUND OF THE INVENTION

The present invention relates to a process for the isolation of nitrated tetracycline derivatives.

Tetracyclines are a group of broad-spectrum antibiotics that contain four hydrocarbon rings. Tetracycline itself was first described in J. Am. Chem. Soc., 1953, 75, 4621. Various tetracycline derivatives have been described since, examples of which are described in U.S. Pat. No. 2,980,584; U.S. Pat. No. 2,990,331; U.S. Pat. No. 3,062,717; U.S. Pat. No. 3,165,531; U.S. Pat. No. 3,454,697; U.S. Pat. No. 3,557,280; U.S. Pat. No. 3,674,859; U.S. Pat. No. 3,957,980; U.S. Pat. No. 4,018,889; U.S. Pat. No. 4,024,272; and U.S. Pat. No. 4,126,680.

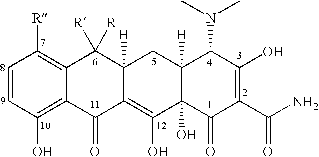

Tetracycline derivatives are chemically stable in strong mineral acids, particularly in concentrated sulfuric acid, and, therefore, can be subjected to certain chemical transformations (J. Am. Chem. Soc., 1960, 82, 1253, J. Med. Chem., 1962, 5(3), 538). Extensive research has been performed on nitration and halogenation of tetracyclines that afforded 7- and/or 9-halo/nitro derivatives.

An effective method of isolating a product from the nitration reaction mixture is precipitation with diethyl ether (GB 876,500; EP 535346; U.S. Pat. No. 5,248,797; U.S. Pat. No. 5,281,628; U.S. Pat. No. 5,401,863). Although ethers are suitable solvents for precipitation of organic salts, diethyl ether being the most widely used for this purpose, such solvents are inconvenient for industrial processes because of related safety issues.

Isolation of minocycline using diethyl ether, according to the prior art, afforded a hardly filterable and very hygroscopic solid, even after washing it on a filter several times with ether. Although suspending the initially isolated product in another portion of ether afforded easily processable non-hygroscopic material, the total amount of ether used for finally isolating the product in a stable dry form was very large.

An alternative isolation process is described in J. Med. Chem., 1962, 5(3), 538, and is performed by diluting the reaction mixture with water followed by extraction with 1-butanol. The desired solid product is then isolated from the organic solution by means of evaporation to dryness. This alternative process is limited to nitrated tetracycline derivatives that are soluble in 1-butanol and is applicable to a limited number of tetracycline derivatives.

WO 2006/130501 discloses, such as in comparative Example 2 and Example 2 thereof, yet another method wherein isopropanol or mixtures of isopropanol and heptane are used to quench the reaction and then additional heptane is added to obtain tigecycline having a suitable purity.

However, there is a need for alternative methods for the isolation of tetracycline derivatives.

### SUMMARY OF THE INVENTION

The present invention provides a process for isolating nitrated tetracycline derivatives from a reaction mixture, which is the result of the nitration of tetracycline derivatives, by precipitation using a C₃-C₈ alcohol without the need for an additional antisolvent. Preferably, the alcohol used is a C₃-C₄ alcohol, even more preferably the alcohol is isopropanol.

### DETAILED DESCRIPTION

The present invention provides a process for isolating nitrated tetracycline derivatives from a reaction mixture, which is the result of the nitration of tetracycline derivatives, by precipitation using only a C₃-C₈alcohol.

In comparison with the prior art, it has been surprisingly found that isopropanol alone is sufficient to precipitatetetracycline derivatives and that the resulting tetracycline derivatives have a chromatographic purity of greater than about 60%, preferably having a chromatographic purity greater than about 77%.

Moreover, it has been found that isolation of the nitrated tetracycline derivatives allows for a dry non-hygroscopic nitration product to be easily obtained.

This process comprises providing a reaction mixture, which is the result of nitration of a tetracycline derivative, combining the reaction mixture with a C₃-C₈ alcohol to obtain a precipitate, and recovering the nitrated tetracycline derivatives.

Nitration of the tetracycline derivative may be performed by any method known in the art, such as described in J. Med. Chem., 1994, 37, 184; U.S. Pat. No. 5,248,797, U.S. Pat. No. 5,401,863.

The nitrating agent used for the nitration of the tetracycline derivative includes, but is not limited to, nitric acid, nitric oxide or metal nitrates. The nitrating agent may be added portionwise to the solution of tetracycline derivative in concentrated or pure acid. This procedure may help in obtaining a higher purity of the product.

The reaction mixture obtained following the nitration reaction may contain a pure acid or concentrated acid solution such as sulfuric acid, methanesulfonic acid, trifluoroacetic acid, trifluoromethanesulfonic acid (triflic acid), phosphoric acid, or perchloric acid.

The reaction mixture may contain, in addition to the tetracycline derivative, nitric acid in concentrated sulfuric acid, metal nitrate in concentrated sulfuric, or nitric acid in acetic anhydride.

In a preferred embodiment of the present invention, the C₃-C₈ alcohol is a, C₃-C₄ alcohol, most preferably isopropanol. Preferably, the tetracycline derivative (weight) to alcohol (volume) ratio ranges from about 1:20 to about 1:120, more preferably from about 1:35 to about 1:90, and most preferably from about 1:50 to about 1:80. Preferably, the sulfuric acid to alcohol (volume) ratio is from about 1:2.5 to about 1:30, more preferably from about 1:4 to about 1:25, and most preferably from about 1:6 to about 1:20.

In a preferred embodiment of the present invention, the tetracycline derivative is selected from 6-demethyltetracycline, bromotetracycline, chlorotetracycline, clomocycline, demeclocycline, doxycycline, lymecycline, meclocycline, methacycline, minocycline, oxytetracycline, rolitetracycline, tetracycline, sancycline, 5a,6-anhydrotetracycline, DDA-tetracycline, dactylocyclinone and 8-methoxychlorotetracycline. The tetracycline derivative also includes the optical isomers of the compounds mentioned above. More preferably, the tetracycline derivative is selected from doxycycline, methacycline, sancycline and minocycline. Even more preferably, the tetracycline derivative is selected from 7-halosancycline, 9-halosancycline and minocycline. Most preferably, the tetracycline derivative is minocycline.

In a preferred embodiment of the present invention, the C₃-C₈ alcohol is introduced in small portions into the reaction mixture until a precipitate appears, whereupon the remainder of the C₃-C₈ alcohol is introduced into the mixture. Most preferably, the alcohol is initially added dropwise to the reaction mixture. This method is preferred as the subsequent filtration of the reaction mixture is faster than known procedures, i.e. pouring the reaction mixture into the organic solvent. Moreover, this procedure enables one to avoid the exothermic heating of the reaction mixture as a result of the addition of an alcohol to a concentrated acid. Indeed, the exothermic nature of the reaction may reduce the purity of the final product.

Introduction of the C₃-C₈ alcohol may be followed by stirring the resulting reaction mixture for about 5 to about 15 hours.

In a preferred embodiment of the present invention, an inert gas is bubbled through the reaction mixture just before and during the addition of the alcohol to the reaction mixture. This procedure increases the product quality by removing nitrogen oxides which may be formed as a result of local heating. The inert gas is preferably nitrogen or argon, most preferably nitrogen.

In a preferred embodiment of the present invention, the nitrated tetracycline derivative is recovered by any method known in the art, such as filtration and drying of the obtained product. Preferably, the filtration temperature is from about 0° C. to about 40° C., more preferably from about 10° C. to about 30° C., and most preferably from about 20° C. to about 25° C.

In a most preferred embodiment of the present invention, filtration of the precipitate obtained after the organic solvent is combined is performed in a nitrogen stream so that the wet solid would not be exposed to air. Hence, when the solid nitration product becomes a pourable powder, it can be stored without special precautions. The obtained product can be dried in an oven, if necessary. Filtration in an inert gas stream also reduces the amount of the alcohol used for washings of the nitrated tetracycline derivative and results in initially dried solid product which is already insensitive to moisture.

Having described the invention with reference to certain preferred embodiments, other embodiments will become apparent to one skilled in the art from consideration of the specification. The invention is further defined by reference to the following examples describing in detail the analysis of the tetracycline derivatives and methods for preparing the tetracycline derivatives of the invention. It will be apparent to those skilled in the art that many modifications, both to materials and methods, may be practiced without departing from the scope of the invention.

### EXAMPLES

### Nitration of minocycline and isolation of crude 9-nitrominocycline

### Example 1—

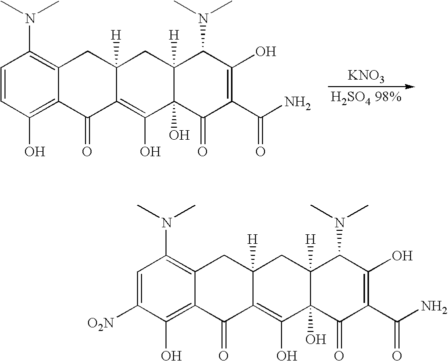 Minocycline hydrochloride (5 g) was completely dissolved in H₂SO₄ 98% (30 ml), whereupon the mixture was cooled to about 0° C.-5° C. and solid KNO3 (1.22 g, 1.2 eq.) was charged into the solution. When the reaction was completed (as determined by HPLC), cold iso-propanol was added dropwise, under cooling at 0° C.-5° C., until a precipitate started to form. The remaining solvent was run continuously into the suspension (total amount of IPA was about 380 ml). The resulting suspension was stirred overnight and filtered under nitrogen in such manner that when all the filtrate was passed through the funnel, the vacuum was disconnected and the solid was further dried in the nitrogen stream. If suitable equipment is available, the filtration can alternatively be performed under nitrogen pressure, i.e. similarly to a common industrial method of filtration. The initially dried material was further dried in a vacuum oven at about 40° C. overnight to afford the desired product as a yellow to brownish solid stable under regular conditions.

### Example 2—

Minocycline hydrochloride (60 gr) was completely dissolved in H₂SO₄ 98% (360 ml), whereupon the mixture was cooled down about 0° C.-5° C. and solid KNO₃ (14.1 gr) was introduced into the solution portion-wise during 1.5-2 hours. When the reaction was complete (based on HPLC), cold iso-propanol was added dropwise, under cooling at 0° C.-5° C., until a precipitate started to form. The remaining solvent was then run continuously into the suspension (total amount of IPA is 3000 ml) . The resulting suspension was stirred for 1 hour and filtered under nitrogen in such manner that when all the filtrate was passed through the funnel, the vacuum was disconnected and the solid was further dried in the nitrogen stream. The initially dried material was further dried in a vacuum oven at about 40° C. overnight to afford 79.4 gr of the desired product, which was characterized by chromatographic purity of about 70.6%.

### Example 3—

Minocycline hydrochloride (5 gr) was completely dissolved in H₂SO₄ 98% (30 ml), whereupon the mixture was cooled down to about 0° C. -5° C. and solid KNO₃ (1.22 gr) was introduced into the solution. When the reaction was complete, (based on HPLC,) cold iso-propanol was added dropwise, under cooling at 0° C.-5° C., until a precipitate started to form. The remaining solvent was run continuously into the suspension (total amount of IPA is 375ml). The resulting suspension was stirred overnight at ambient temperature, and then filtered and washed twice with about 20 mL portions of IPA under nitrogen. When all the filtrate was passed through the funnel, the vacuum was disconnected and the solid was further dried in the nitrogen stream. The initially dried material was further dried in a vacuum oven at about 40° C. overnight to afford 7.77 gr of the desired product, which was characterized by chromatographic purity of about 77%.

1. A process for isolating nitrated tetracycline derivatives from a reaction mixture by precipitation using C₃-C₈ alcohols comprising:
a. providing a reaction mixture resulting from the nitration of a tetracycline derivative;
b. combining said reaction mixture with a solvent consisting of a C₃-C₈alcohol to obtain a precipitate; and
c. recovering said nitrated tetracycline derivative.

2. The process of claim 1, wherein said C₃-C₈alcohol is a C₃-C₄ alcohol.

3. The process of claim 2, wherein said C₃-C₄ alcohol is isopropanol.

4. The process claim 1, wherein said tetracycline derivative (weight) to alcohol (volume) ratio is from about 1:20 to about 1:120.

5. The process of claim 4, wherein said tetracycline derivative (weight) to alcohol (volume) ratio is from about 1:35 to about 1:90.

6. The process of claim 5, wherein said tetracycline derivative (weight) to alcohol (volume) ratio is from about 1:50 to about 1:80.

7. The process of claim 1, wherein said tetracycline derivative is selected from the group consisting of doxycycline, methacycline, sancycline, and minocycline.

8. The process of claim 7, wherein said tetracycline derivative is selected from the group consisting of: 7-halosancycline, 9-halosancycline, and minocycline.

9. The process of claim 8, wherein said tetracycline derivative is minocycline.

10. The process of claim 1, wherein said isolated nitrated tetracycline derivatives have a chromographic purity of greater than about 60%

11. The process of claim 10, wherein said isolated nitrated tetracycline derivatives have a chromographic purity of greater than about 77%.

12. The process of claim 1,further comprising the step of stirring said alcohol after said combination step.

13. The process of claim 12, wherein said stirring is for about 5 to about 15 hours.
